(19) United States
(12) Patent Application Publication
(10) Pub. No.: US 2023/0335278 A1
(43) Pub. Date: Oct. 19, 2023

(54) DIAGNOSIS ASSISTANCE APPARATUS, DIAGNOSIS ASSISTANCE METHOD, AND COMPUTER READABLE RECORDING MEDIUM

(71) Applicant: NEC Corporation, Minato-ku, Tokyo (JP)

(72) Inventor: Kazuhisa TAKAGI, Tokyo (JP)

(73) Assignee: NEC Corporation, Minato-ku, Tokyo (JP)

(21) Appl. No.: 18/026,558

(22) PCT Filed: Sep. 28, 2020
(86) PCT No.: PCT/JP2020/036695

§ 371 Date: Mar. 15, 2023

(51) Int. Cl. G16H 50/20

(52) U.S. Cl. CPC G16H 50/20; G16H 10/60

(57) ABSTRACT

A diagnosis assistance apparatus includes: a learning model selection unit that selects a first learning model in accordance with a patient to be diagnosed, the first learning model indicating a relationship between waveforms of an electrocardiogram and a disease; an estimation unit that estimates, using the selected first learning model, a possibility of a disease of the patient to be diagnosed based on electrocardiogram data of the patient to be diagnosed; and a presentation unit that presents a result of the estimation and an evidence for the result of the estimation.

### TECHNICAL FIELD

The present invention relates to a diagnosis assistance apparatus and a diagnosis assistance method for assisting a doctor in making a diagnosis of a cardiac disease, and further relates to a computer readable recording medium in which a program for realizing these apparatus and method has been recorded.

### BACKGROUND ART

An electrocardiogram is a recording of the condition of the electrical activity of the heart of a patient as a graph. A doctor reads waveforms recorded on the electrocardiogram, and makes a diagnosis of a cardiac disease of the patient from the waveforms.

However, it is not easy to find an abnormality from the electrocardiogram, and the finding also depends on the doctors’ skills, which gives rise to the possibility that there are differences in the diagnosis results. In view of this, patent document 1 discloses an analysis apparatus that analyzes an electrocardiogram and outputs the result of the analysis.

Specifically, the analysis apparatus disclosed in patent document 1 obtains electrocardiogram data of a patient, and then divides the obtained electrocardiogram data into pieces of waveform data on a per-heartbeat basis. Next, the analysis apparatus disclosed in patent document 1 categorizes the individual pieces of waveform data based on a pre-set categorization condition, and generates groups of waveforms with similar features. Thereafter, the analysis apparatus disclosed in patent document 1 performs statistical processing with respect to the groups of waveforms, derives such statistical values as the number of abnormal heartbeats, the ratio of this number to the total number of heartbeats, and the maximum and minimum heart rates for each group of waveforms, adds information of the patient to the obtained statistical values, and outputs the result of the addition as the result of the analysis.

### LIST OF RELATED ART DOCUMENTS

### Patent Document

Patent document 1: Japanese Patent Laid-Open Publication No. 2007-20799

### SUMMARY OF INVENTION

### Problems to Be Solved by the Invention

However, the analysis apparatus disclosed in patent document 1 does not present the possibility of a cardiac disease in view of information of the patient. Therefore, even if doctors made a diagnosis of cardiac diseases using the results of the analysis provided by this apparatus, there is a possibility that there are differences in the diagnosis results.

An example object of the present invention is to provide a diagnosis assistance apparatus, a diagnosis assistance method, and a computer-readable recording medium that solve the aforementioned problem, and present diagnostic materials based on information of a patient to be diagnosed in diagnosing a cardiac disease of a patient.

### Means for Solving the Problems

In order to achieve the above-described object, a diagnosis assistance apparatus includes:
a learning model selection unit that selects a first learning model in accordance with a patient to be diagnosed, the first learning model indicating a relationship between waveforms of an electrocardiogram and a disease;
an estimation unit that estimates, using the selected first learning model, a possibility of a disease of the patient to be diagnosed based on electrocardiogram data of the patient to be diagnosed; and
a presentation unit that presents a result of the estimation and an evidence for the result of the estimation.

In addition, in order to achieve the above-described object, a diagnosis assistance method includes:
a learning model selection step of selecting a first learning model in accordance with a patient to be diagnosed, the first learning model indicating a relationship between waveforms of an electrocardiogram and a disease;
an estimation step of estimating a possibility of a disease of the patient to be diagnosed based on electrocardiogram data of the patient to be diagnosed, using the selected first learning model,; and
a presentation step of presenting a result of the estimation and an evidence for the result of the estimation.

Furthermore, in order to achieve the above-described object, a first computer readable recording medium according to an example aspect of the invention is a computer readable recording medium that includes recorded thereon a program, the program including instructions that cause a computer to carry out:
a learning model selection step of selecting a first learning model in accordance with a patient to be diagnosed, the first learning model indicating a relationship between waveforms of an electrocardiogram and a disease;
an estimation step of estimating a possibility of a disease of the patient to be diagnosed based on electrocardiogram data of the patient to be diagnosed, using the selected first learning model; and
a presentation step of presenting a result of the estimation and an evidence for the result of the estimation.

### Advantageous Effects of the Invention

As described above, according to the invention, it is possible to present diagnostic materials based on information of a patient to be diagnosed in diagnosing a cardiac disease of a patient.

### BRIEF DESCRIPTION OF THE DRAWINGS

FIG. 1 is a configuration diagram showing the schematic configuration of the diagnosis assistance apparatus according to the example embodiment.

FIG. 2 is a diagram illustrating respective waves in an electrocardiogram.

FIG. 3 is a configuration diagram specifically showing the configuration of the diagnosis assistance apparatus according to the example embodiment.

FIG. 4 shows one example of medical record data of a patient used in the example embodiment.

FIG. 5 shows one example of electrocardiogram data of a patient used in the example embodiment.

FIG. 6 is a diagram showing examples of electrocardiogram data and labels that are used as the training data in the example embodiment.

FIG. 7 is a diagram that conceptually illustrates the functions of the second learning model (selection model) used in the example embodiment.

FIG. 8 is a flow diagram showing the overall operations during the machine learning processing in the diagnosis assistance apparatus according to the example embodiment.

FIG. 9 is a flow diagram specifically showing step A2 shown in FIG. 8 (processing for training the selection model).

FIG. 10 is a diagram illustrating processing in step A21 shown in FIG. 9.

FIG. 11 is a diagram illustrating processing in step A22 shown in FIG. 9.

FIG. 12 is a flow diagram specifically showing step A3 shown in FIG. 8 (processing for training the disease estimation models).

FIG. 13 is a diagram illustrating processing in step A33 shown in FIG. 12.

FIG. 14 is a flow diagram showing the operations during the diagnosis assistance processing in the diagnosis assistance apparatus according to the example embodiment.

FIG. 15 is a diagram illustrating processing in respective steps shown in FIG. 14.

FIG. 16 is a diagram showing an example of information presented to a user in the example embodiment.

FIG. 17 is a block diagram illustrating an example of a computer that realizes the diagnosis assistance apparatus according to the example embodiment.

### EXAMPLE EMBODIMENT

### Example Embodiment

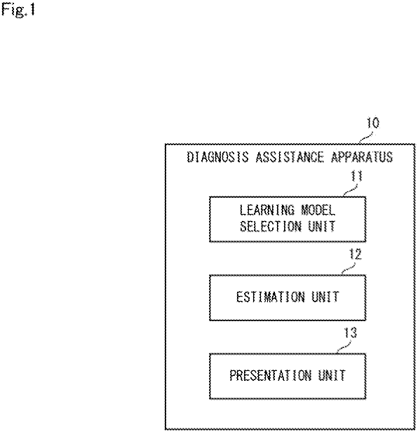

The following describes a diagnosis assistance apparatus, a diagnosis assistance method, and a program according to the present example embodiment with reference to FIG. 1 to FIG. 17.

### [Apparatus Configuration]

First, a schematic configuration of the diagnosis assistance apparatus according to the example embodiment will be described using FIG. 1. FIG. 1 is a configuration diagram showing the schematic configuration of the diagnosis assistance apparatus according to the example embodiment.

A diagnosis assistance apparatus 10 according to the example embodiment shown in FIG. 1 is an apparatus for assisting a doctor in making a diagnosis of a cardiac disease using electrocardiogram data of a patient. As shown in FIG. 1, the diagnosis assistance apparatus 10 includes a learning model selection unit 11, an estimation unit 12, and a presentation unit 13.

The learning model selection unit 11 selects a first learning model that indicates a relationship between waveforms of an electrocardiogram and a disease in accordance with a patient who is to be diagnosed. In the example embodiment, the learning model selection unit 11 selects any of a plurality of trained models/estimation models as the first learning model based on information of the patient to be diagnosed. The first learning model is, for example, a machine-trained model related to a relationship between electrocardiogram data and a disease, which has been generated in accordance with the attributes of the patient. Hereinafter, the first learning model is referred to as a “disease estimation model”. Examples of the information of the patient include personal information of the patient, such as medical record data, biological data, and attribute information.

Using the disease estimation model selected by the learning model selection unit 11, the estimation unit 12 estimates the possibility of a disease of the patient to be diagnosed based on electrocardiogram data of that patient. The presentation unit 13 presents the estimation result achieved by the estimation unit 12 and the evidence based on which the estimation result was derived.

As described above, the diagnosis assistance apparatus 10 selects a learning model appropriate for a patient to be diagnosed from information of that patient, such as the age, sex, previous medical history, family history, and smoking history, and estimates the possibility of a disease by applying electrocardiogram data of that patient to the selected learning model. That is to say, in diagnosing a cardiac disease of a patient, the diagnosis assistance apparatus 10 can present diagnostic materials based on information of a patient to be diagnosed.

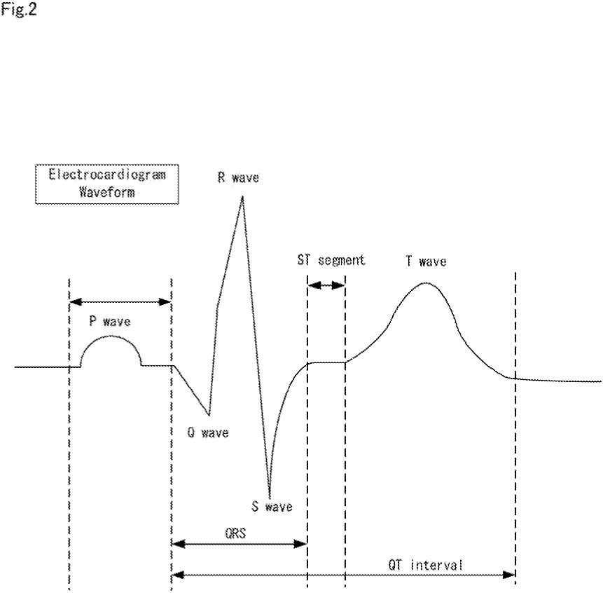

Using FIG. 2, a description is now given of a general diagnosis of a cardiac disease that is made by a doctor with use of electrocardiogram data. FIG. 2 is a diagram illustrating respective waves in an electrocardiogram. As shown in FIG. 2, the electrocardiogram normally includes characteristic waveforms, such as a P wave, a Q wave, an R wave, an S wave, a T wave, and an ST segment. A doctor reads the P wave, Q wave, R wave, S wave, T wave, and ST section from the electrocardiogram, and makes a diagnosis of a cardiac disease by finding an abnormality from the status of each wave. For example, if the T wave is flatter than normal or downward relative to a base line, the doctor diagnoses that a patient has a possibility of having an ischemic cardiac disease (angina and myocardial infarction) while looking up information of the medical record of the patient.

In contrast, when the diagnosis assistance apparatus 10 is used, the possibility of a disease in view of information (the medical record) of a patient to be diagnosed, as well as the evidence thereof, is presented based on electrocardiogram data with use of an analysis model that has been selected in accordance with that patient. This reduces the possibility that differences arise in the results of diagnoses made by doctors.

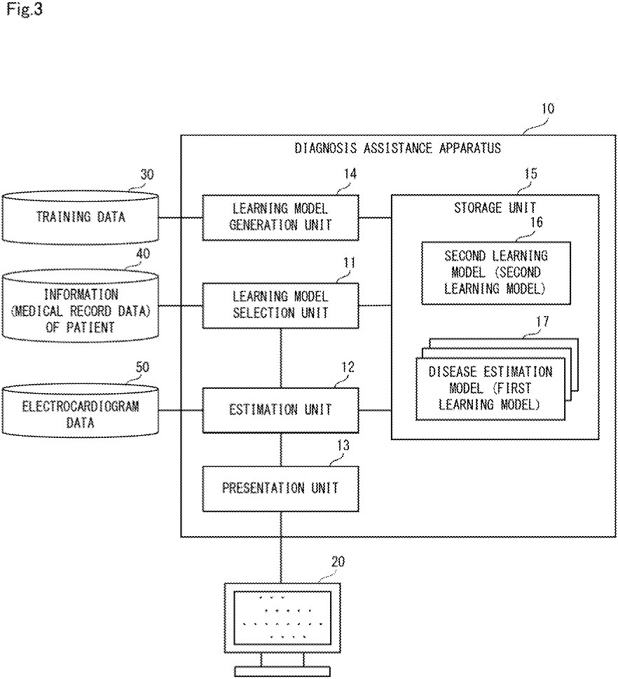

Next, the configuration and functions of the diagnosis assistance apparatus 10 according to the example embodiment will be described specifically using FIG. 3 to FIG. 6. FIG. 3 is a configuration diagram specifically showing the configuration of the diagnosis assistance apparatus according to the example embodiment.

As shown in FIG. 3, in the example embodiment, the diagnosis assistance apparatus 10 includes a learning model generation unit 14 and a storage unit 15, in addition to the learning model selection unit 11, estimation unit 12, and presentation unit 13 that have been described earlier. Also, as shown in FIG. 3, a display apparatus 20 is connected to the diagnosis assistance apparatus 10.

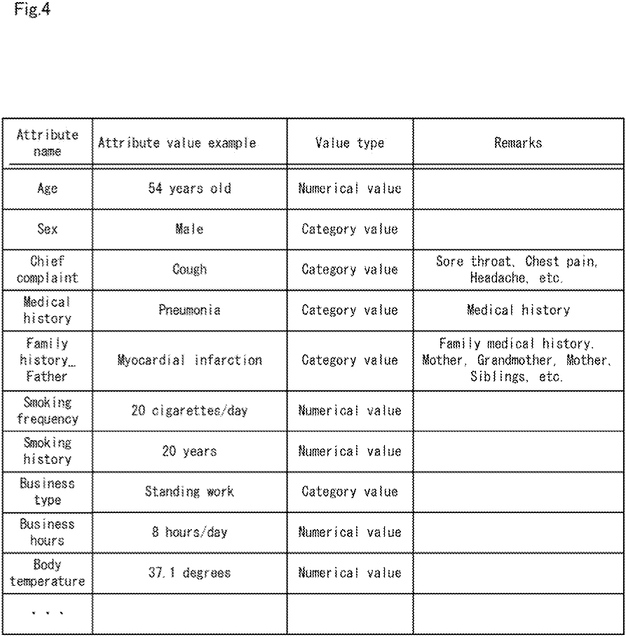

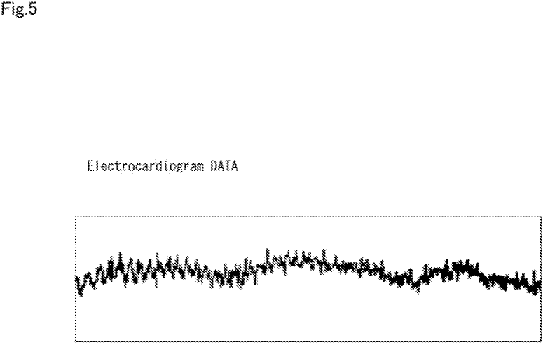

Furthermore, although not shown in FIG. 3, the diagnosis assistance apparatus 10 is connected to an external apparatus via a network in such a manner that they can perform data communication. The external apparatus transmits training data 30 to be used by the learning model generation unit 14, information (e.g., medical record data) 40 of a patient to be diagnosed, and electrocardiogram data 50 of the patient to be diagnosed to the diagnosis assistance apparatus 10. FIG. 4 shows one example of medical record data of a patient used in the example embodiment. FIG. 5 shows one example of electrocardiogram data of a patient used in the example embodiment.

The learning model generation unit 14 generates disease estimation models 17 by performing machine learning with use of the training data 30. The method of machine learning is not limited in particular. Examples of the method of machine learning include deep learning.

Examples of the training data include information of patients, electrocardiogram data of patients, and labels indicating the diseases corresponding to the electrocardiogram data (hereinafter referred to as “ground truth labels”) that have been obtained in advance. Note that “patients” associated with the training data are patients from whom the training data has been obtained. Furthermore, examples of the information of patients to be used as the training data include the medical record data shown in FIG. 4, biological information, and attribute information. Examples of the electrocardiogram data of patients to be used as the training data include the electrocardiogram data shown in FIG. 5.

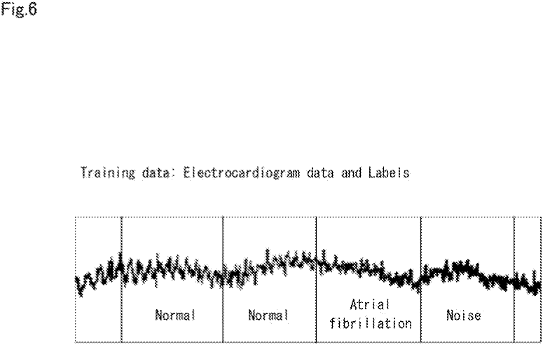

Examples of the ground truth labels indicating the diseases corresponding to electrocardiogram data include labels that are respectively added to sections of electrocardiogram data as shown in FIG. 6. FIG. 6 is a diagram showing examples of electrocardiogram data and labels that are used as the training data in the example embodiment. In the examples of FIG. 6, the sections are set by dividing the electrocardiogram data at a predetermined time interval. Hereinafter, each section is referred to as a piece of “partial electrocardiogram data”. Also, in the examples of FIG. 6, “normal”, “atrial fibrillation”, or “noise” is set as a ground truth label for each section of the electrocardiogram data. Examples of the labels are not limited to the foregoing examples, and also include “bigeminal pulse”, “arrhythmia”, “myocardial infarction”, “angina”, and so forth. Furthermore, the labels may be finely categorized. For example, the categories of angina include effort angina, unstable angina, vasospastic angina (variant angina), angina caused by arteriosclerosis, asymptomatic myocardial ischemia, and the like.

Furthermore, in the example embodiment, the learning model generation unit 14 first inputs electrocardiogram data of patients included in the training data 30 to the disease estimation models 17, and obtains the output results. Then, the learning model generation unit 14 performs machine learning while using the obtained output results, information of the patients, and the ground truth labels indicating the diseases corresponding to the electrocardiogram data as training data, and generates a second learning model 16 indicating a correspondence relationship between the information (medical record data) of the patients and the disease estimation models.

As will be described later, the second learning model 16 is used by the learning model selection unit 11 in selecting a disease estimation model 17. Hereinafter, the second learning model is referred to as a “selection model”. The method of machine learning in this case, too, is not limited in particular. Examples of the method of machine learning include deep learning.

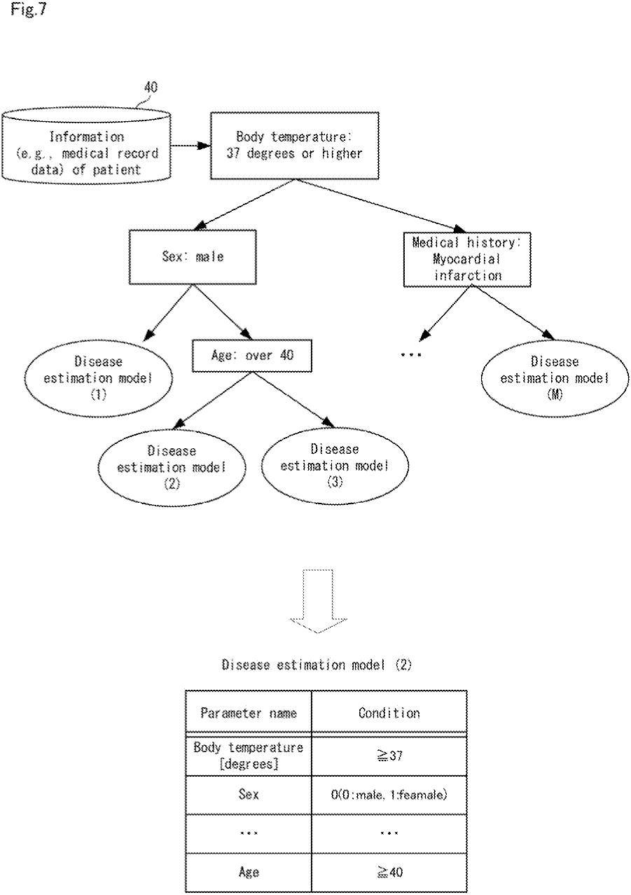

FIG. 7 is a diagram that conceptually illustrates the functions of the second learning model (selection model) used in the example embodiment. As shown in FIG. 7, once, for example, medical record data has been input to the selection model 16 as information of a patient, a disease estimation model 17 is selected from among disease estimation models (1) to (M) in accordance with the contents of the medical record data. M indicates the number of disease estimation models that have been prepared.

Furthermore, the learning model generation unit 14 can update the disease estimation model 17 using the selection model 16. Specifically, the learning model generation unit 14 first inputs information of individuals to be used as the training data to the selection model 16, and specifies a corresponding disease estimation model for each individual. Then, the learning model generation unit 14 selects electrocardiogram data and a ground truth label that correspond to the specified disease estimation model from among electrocardiogram data of patients and the ground truth labels indicating the diseases corresponding to the electrocardiogram data, which are used as the training data. Thereafter, the learning model generation unit 14 updates the disease estimation model using the selected electrocardiogram data and ground truth label.

Specifically, once the learning model generation unit 14 has specified a disease estimation model for each patient from whom training data has been obtained, it allocates electrocardiogram data and a ground truth label to be used as the training data for each patient. Then, for each patient, the learning model generation unit 14 inputs the allocated electrocardiogram data to the specified disease estimation model, compares the output results with the ground truth label, and updates the disease estimation model based on the comparison result.

In the example embodiment, using the above-described selection model, the learning model selection unit 11 selects a disease estimation model that fits a patient to be diagnosed from among the disease estimation models that have been generated in advance based on information of the patient to be diagnosed.

In the example embodiment, the estimation unit 12 analyzes the possibility of a disease of the patient to be diagnosed based on the output results of the disease estimation model that respectively correspond to pieces of partial electrocardiogram data obtained by dividing the electrocardiogram data of the patient to be diagnosed at the predetermined time interval. Specifically, the estimation unit 12 inputs each piece of partial electrocardiogram data to the disease estimation model, and obtains the output results. Then, using the output results that respectively correspond to the pieces of partial electrocardiogram data, the estimation unit 12 estimates the possibility of a disease of the patient to be diagnosed.

In the example embodiment, the presentation unit 13 presents the estimation result and the evidence based on which the estimation result was derived on a screen of the display apparatus 20. Examples of the estimation result that is presented at this time include information of a disease that has a possibility of being present in the patient to be diagnosed. Also, examples of the evidence that is presented at this time include the reason for specification of the disease that has a possibility of being present in the patient to be diagnosed.

Furthermore, the evidence may be at least one of a partial electrocardiogram data (a specific portion) and the attributes of the patient. Moreover, examples of the attributes include the attributes of the patient corresponding to the selected disease estimation model. In addition, after presenting the evidence, the presentation unit 13 can also present the estimation result in accordance with a request for presenting the estimation result in connection with the presented evidence. Also, the evidence may be at least one of pieces of data that have been input to the disease estimation model. Examples of the evidence also include a partial waveform included in electrocardiogram waveform data, the attributes of the patient to be diagnosed, and so forth.

### [Apparatus Operations]

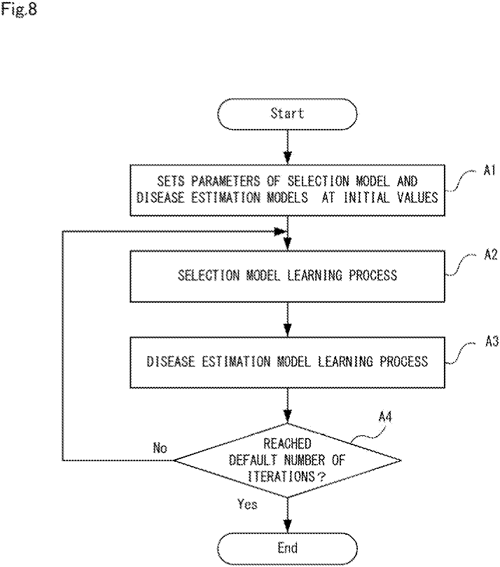

Using FIG. 8 to FIG. 16, the following provides a description of the operations of the diagnosis assistance apparatus 10 according to the example embodiment, which are grouped into machine learning processing and diagnosis assistance processing. In the following description, FIG. 1 to FIG. 7 will be referred to as appropriate. Also, in the example embodiment, the diagnosis assistance method is implemented by causing the diagnosis assistance apparatus 10 to operate. Therefore, the following description of the operations of the diagnosis assistance apparatus 10 applies to the diagnosis assistance method according to the example embodiment.

### Learning Model Generation Processing:

First, processing for generating learning models, which is performed by the diagnosis assistance apparatus 10, will be described using FIG. 8 to FIG. 10. FIG. 8 is a flow diagram showing the overall operations during the machine learning processing in the diagnosis assistance apparatus according to the example embodiment.

The example embodiment is based on the precondition that, beforehand, medical record data is generated by taking a history from a patient to be diagnosed, and furthermore, electrocardiogram data is obtained by taking an electrocardiogram of the patient to be diagnosed, in order to obtain training data 30. In addition, a doctor makes a diagnosis with respect to the electrocardiogram data, and a ground truth label is set for each section (see FIG. 6). That is to say, in the training data, a ground truth label indicating “normal”, “atrial fibrillation”, “bigeminal pulse”, “noise”, or the like is added on a per-section basis.

As shown in FIG. 8, first, the learning model generation unit 14 sets parameters of a model used as the selection model 16 and parameters of models used as the disease estimation models 17 at their respective initial values (step A1).

Next, the learning model generation unit 14 executes machine learning with respect to the selection model indicating a correspondence relationship between medical record data and the disease estimation models (step A2). Specifically, the learning model generation unit 14 inputs electrocardiogram data included in the training data 30 to the disease estimation models 17, and obtains the output results. Then, the learning model generation unit 14 updates the parameters of the selection model 16 by executing machine learning while using the obtained output results and the ground truth labels included in the training data 30 as training data.

Next, the learning model generation unit 14 executes machine learning in order to generate the disease estimation models 17 that indicate the relationships between waveforms of electrocardiograms and diseases in accordance with patients (step A3). Specifically, the learning model generation unit 14 updates the parameters of the disease estimation models 17 by executing machine learning while using medical record data of the patients, electrocardiogram data of the patients, and the ground truth labels as training data.

Next, the learning model generation unit 14 determines whether the number of times steps A2 and A3 have been executed has reached a predetermined number of iterations (step A4). In a case where the result of the determination in step A4 shows that the number of times steps A2 and A3 have been executed has not reached the predetermined number of iterations, the learning model generation unit 14 executes step A2 again. On the other hand, in a case where the number of times steps A2 and A3 have been executed has reached the predetermined number of iterations, the learning model generation unit 14 ends the machine learning processing.

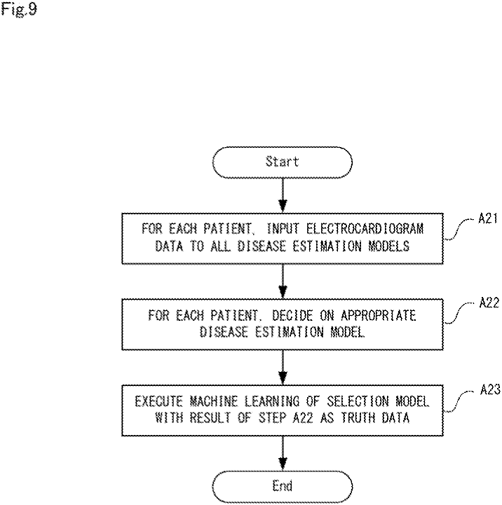

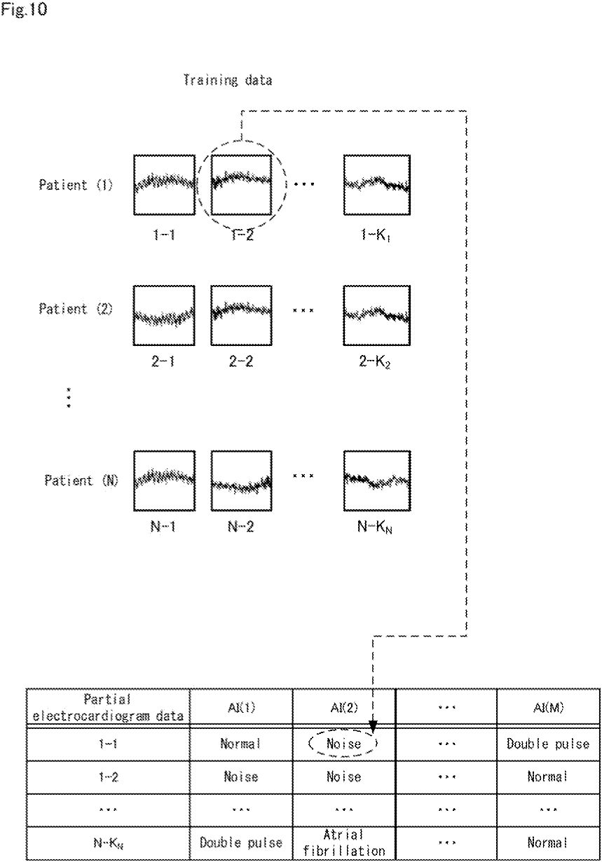

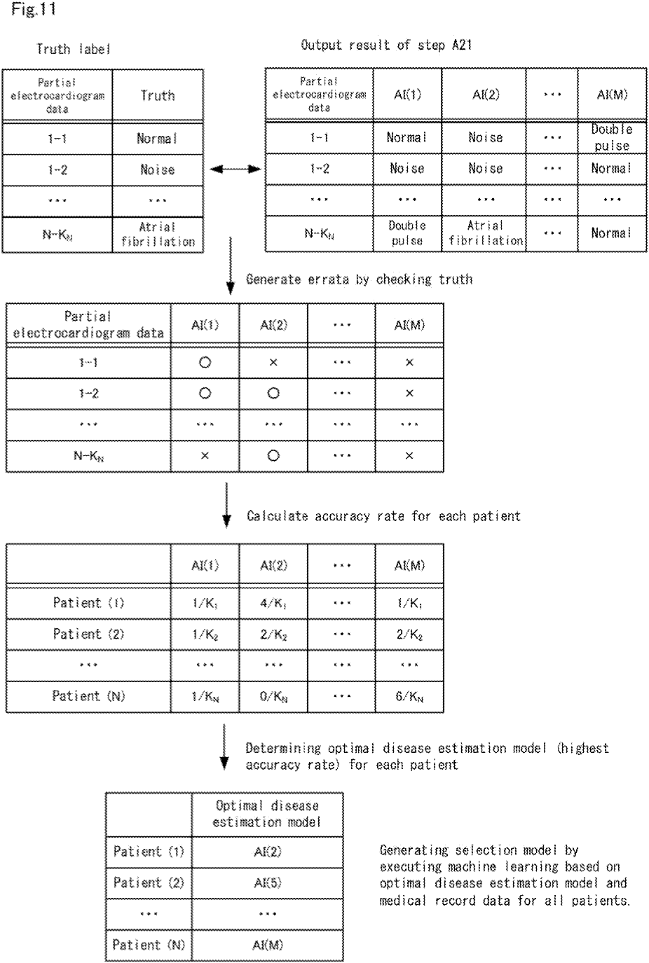

Next, processing of step A2 shown in FIG. 8 (processing for training the selection model) will be described specifically using FIG. 9 to FIG. 11. FIG. 9 is a flow diagram specifically showing step A2 shown in FIG. 8 (processing for training the selection model). FIG. 10 is a diagram illustrating processing in step A21 shown in FIG. 9. FIG. 11 is a diagram illustrating processing in step A22 shown in FIG. 9.

As shown in FIG. 9, for each patient from whom training data 30 has been obtained, the learning model generation unit 14 inputs his/her electrocardiogram data to all disease estimation models 17 (step A21).

For example, as shown in FIG. 10, for each of a patient (1) to a patient (N) from whom training data has been obtained, the learning model generation unit 14 inputs his/her electrocardiogram data to a disease estimation model (1) to a disease estimation model (M). In this way, as shown in the bottom tier of FIG. 10, the output results of all disease estimation models 17 are obtained for all sections of all pieces of electrocardiogram data.

Also, in FIG. 10, the disease estimation models are denoted by “AI”. K sections are set in electrocardiogram data of each patient in advance. “1-1” to “N-K_{N}″ shown in FIG. 10 denote section IDs (Identifiers).

Next, for each patient from whom training data has been obtained, the learning model generation unit 14 decides on an appropriate disease estimation model 17 based on the output results (step A22).

Specifically, in step A22, for each patient from whom training data has been obtained, the learning model generation unit 14 specifies a disease estimation model that includes a large number of sections with a correct estimation result from among all disease estimation models 17. Then, the learning model generation unit 14 decides the specified disease estimation model as a disease estimation model appropriate for that patient.

A description is now given of an example of processing of step A22. As shown in FIG. 11, the learning model generation unit 14 compares ground truth labels with the output results of step A21. Next, the learning model generation unit 14 creates tables indicating right or wrong respectively for the disease estimation models 17 in units of sections of electrocardiogram data, and calculates the accuracy rate of each disease estimation model 17 for each patient using the created tables indicating right or wrong. Next, the learning model generation unit 14 specifies a disease estimation model 17 with the highest accuracy rate for each patient, and decides the specified disease estimation model 17 as a disease estimation model 17 appropriate for that patient. Note that processing of step A22 is not limited to the above description, and a disease estimation model may be decided on based on a predetermined rule that has been set in advance (e.g., the attributes, biological information, and medical record data of the patient).

Next, the learning model generation unit 14 uses the disease estimation model of each patient that was decided on in step A22 as ground truth data, and updates parameters of the selection model 16 by performing machine learning while using this ground truth data and medical record data as training data (step A23). As a result, the selection model 16 is generated.

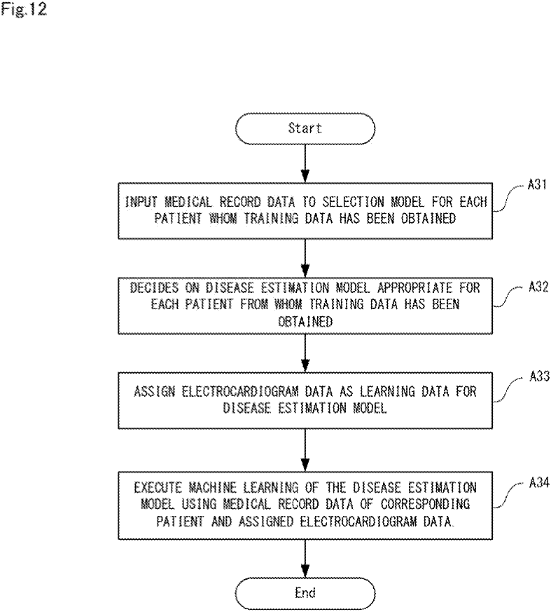

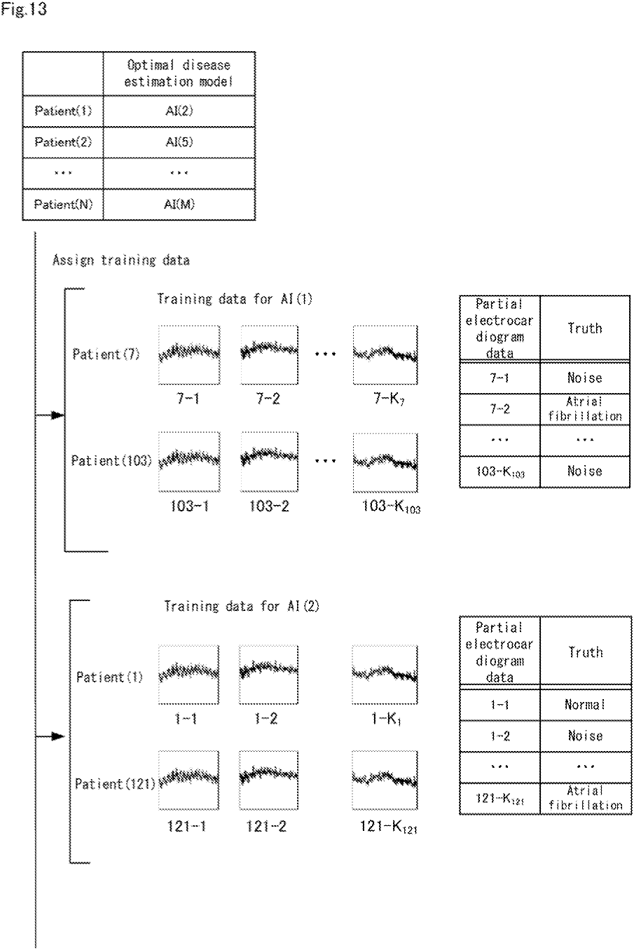

Next, processing of step A3 shown in FIG. 8 (processing for training the disease estimation models) will be described specifically using FIG. 12 and FIG. 13. FIG. 12 is a flow diagram specifically showing step A3 shown in FIG. 8 (processing for training the disease estimation models). FIG. 13 is a diagram illustrating processing in step A33 shown in FIG. 12.

As shown in FIG. 12, first, for each patient from whom training data has been obtained, the learning model generation unit 14 inputs his/her medical record data to the selection model 16 (step A31).

Next, for each patient from whom training data has been obtained, the learning model generation unit 14 decides on a disease estimation model 17 appropriate for that patient based on the output results from the selection model 16 (step A32).

Next, for each patient from whom training data has been obtained, the learning model generation unit 14 assigns electrocardiogram data of that patient as learning data corresponding to the disease estimation model that has been decided on (step A33).

Specifically, in the example of FIG. 13, the learning model generation unit 14 specifies corresponding patients for each disease estimation model 17, and assigns, for each disease estimation model 17, electrocardiogram data of patients corresponding to the disease estimation model 17. In the example of FIG. 13, with respect to the disease estimation model (1), pieces of electrocardiogram data 7-1 to 7-K₇ of a corresponding patient (7), as well as pieces of electrocardiogram data 103-1 to 103-K₁₀₃ of a similarly corresponding patient (103), are assigned.

Next, the learning model generation unit 14 updates parameters of the disease estimation models 17 by performing machine learning for each disease estimation model while using medical record data of corresponding patients, as well as electrocardiogram data assigned in step A33, as training data (step A34). As a result, the disease estimation models 17 are generated.

### Diagnosis Assistance Processing:

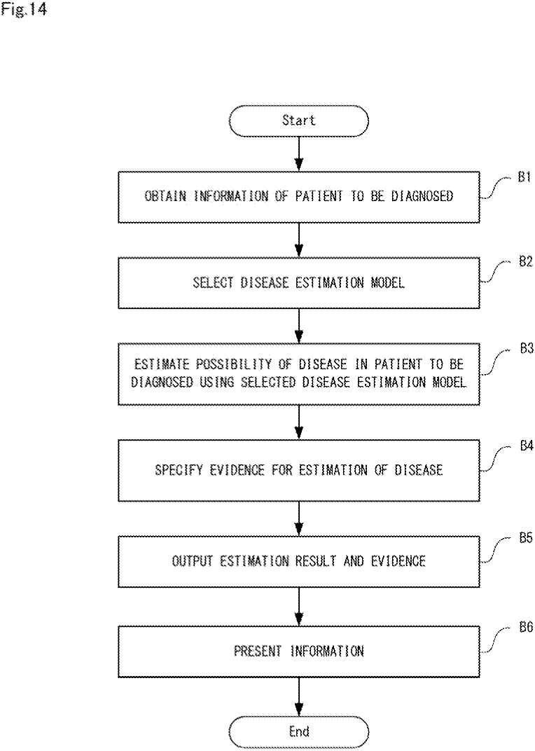

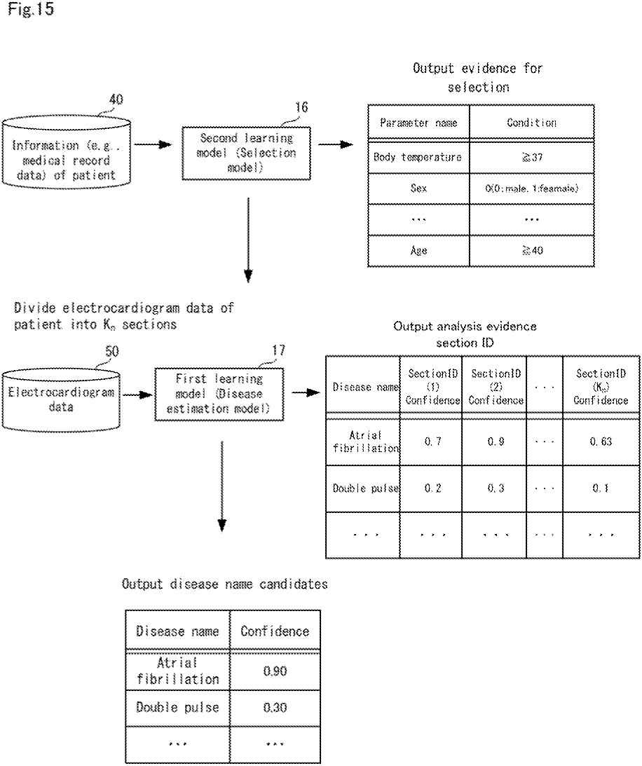

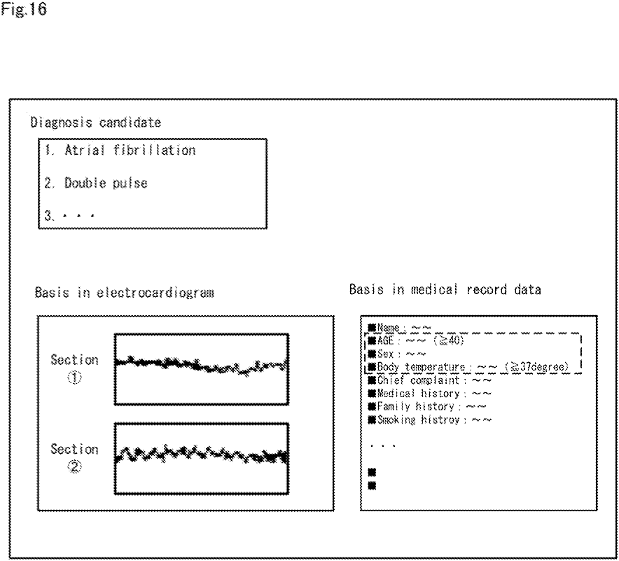

The diagnosis assistance processing performed by the diagnosis assistance apparatus 10 will be described using FIG. 14 to FIG. 16. FIG. 14 is a flow diagram showing the operations during the diagnosis assistance processing in the diagnosis assistance apparatus according to the example embodiment. FIG. 15 is a diagram illustrating processing in respective steps shown in FIG. 14. FIG. 16 is a diagram showing an example of information presented to a user in the example embodiment.

As shown in FIG. 14, first, the learning model selection unit 11 obtains information (e.g., medical record data) 40 of a patient to be diagnosed (step B1). Also, the learning model selection unit 11 obtains electrocardiogram data of the person to be diagnosed.

Next, based on the information 40 of the patient obtained in step B1, the learning model selection unit 11 selects a disease estimation model 16 for estimating the possibility of a disease from electrocardiogram data of the patient to be diagnosed. Specifically, based on the output results of step B1, the learning model selection unit 11 selects a disease estimation model 17 corresponding to the patient from among the disease estimation models 17 that have been generated in advance (step B2).

Also, in step B2, the learning model selection unit 11 outputs the selected disease estimation model 17 and information that serves as the evidence for the selection to the estimation unit 12. As shown in FIG. 15, the information that serves as the evidence for the selection is parameters that have been used in the selection of the disease estimation model 17 and the values thereof (see FIG. 7).

Next, the estimation unit 12 inputs electrocardiogram data 50 of the patient to be diagnosed to the disease estimation model 17 selected in step B2, and estimates the possibility of a disease of the patient (step B3). Also, the electrocardiogram data to be diagnosed is input in a state where it has been divided into, for example, Kₙ sections (see FIG. 15).

For example, in step B3, the estimation unit 12 sets the sections by dividing the electrocardiogram data 50 at the predetermined time interval. Then, as shown in FIG. 15, the estimation unit 12 inputs each of pieces of partial electrocardiogram data obtained by setting the sections to the disease estimation model 17, and calculates a certainty degree indicating the possibility of a disease on a per-section basis.

In the example of FIG. 15, for the section ID (1), a certainty degree of 0.7 is calculated with respect to atrial fibrillation, and a certainty degree of 0.2 is calculated with respect to bigeminal pulse. Also, an output result obtained through general deep learning may be used as a certainty degree. The method of calculating a certainty degree is not limited in particular.

Furthermore, using the certainty degrees of respective diseases in each section, the estimation unit 12 calculates an overall certainty degree with respect to each disease as the possibility of the disease for the patient. Specifically, the estimation unit 12 may use the highest certainty degree among the certainty degrees in the respective sections as the overall certainty degree, or may use an average value of the certainty degrees in respective sections as the overall certainty degree. Furthermore, in a case where the average value is calculated as the overall certainty degree, only several high certainty degrees with large values may be used. The method of calculating the overall certainty degree, too, is not limited in particular.

Next, the estimation unit 12 specifies an evidence for the estimation of the disease in step B3 based on the information that serves as the evidence for the selection, which was output from the learning model selection unit 11 (step B4). Specifically, in connection with a cardiac disease for which the certainty degree is equal to or higher than a threshold, the estimation unit 12 specifies a section in which the certainty degree is equal to or higher than a certain value as the evidence.

Next, the estimation unit 12 outputs the possibility of the disease estimated in step B3, as well as the evidence specified in step B4, to the presentation unit 13 (step B5). Specifically, the estimation unit 12 outputs the name of the cardiac disease for which the certainty degree is equal to or higher than the threshold, as well as the section ID of the section in which the certainty degree indicating the possibility of that cardiac disease is equal to or higher than the certain value, to the presentation unit 13.

Next, the presentation unit 13 presents the estimation result output in step B3 and the evidence on the screen of the display apparatus 20 (step B6). Furthermore, the presentation unit 13 can also present “the information that serves as the evidence for the selection”, which was output to the estimation unit 12 in step B2, on the screen of the display apparatus 20.

For example, as shown in the example of FIG. 16, the presentation unit 13 displays the names of cardiac diseases for which the certainty degree is equal to or higher than the threshold as candidate for the diagnosis result, and displays the pieces of partial electrocardiogram data in the sections in which the certainty degree is equal to or higher than the certain value as the locations of the evidence in the electrocardiogram, on the screen of the display apparatus 20.

Also, in the example of FIG. 16, the presentation unit 13 further displays “the information that serves as the evidence for the selection”, which was output in step B3, as the location of the evidence in medical record on the screen of the display apparatus 20. Furthermore, in the example of FIG. 16, once the user has selected the name of one cardiac disease on the screen, the presentation unit 13 displays the piece of partial electrocardiogram data in the section corresponding to the selected cardiac disease.

Note that the displayed evidences are not limited to the ones described above. For example, the evidences may be the names of items included in medical record data of a target patient, biological information of a target patient, and the like; the evidences may be other than these and not limited to these as long as they are information used in estimating a disease.

### [Effects of Embodiment]

As described above, according to the example embodiment, the estimation is performed using the medical record and electrocardiogram of a patient, and the possibility of a cardiac disease is presented as the estimation result. Furthermore, according to the example embodiment, the evidence based on which the estimation result has been derived, as well as the related portion of the medical record of the patient, is also presented. Therefore, according to the example embodiment, in diagnosis of a cardiac disease of a patient using an electrocardiogram, diagnostic materials based on the medical record of the patient can be presented, and differences in the results of diagnoses of cardiac diseases are reduced.

### [Program]

It suffices for a program in the example embodiment to be a program that causes a computer to carry out steps A1 to A4 shown in FIG. 8 and steps B1 to B6 shown in FIG. 14. Also, by this program being installed and executed in the computer, the diagnosis assistance apparatus and the diagnosis assistance method according to the example embodiment can be realized. In this case, a processor of the computer functions and performs processing as the learning model selection unit 11, the estimation unit 12, the presentation unit 13, and the learning model generation unit 14.

In the example embodiment, the storage unit 15 may be realized by storing data files constituting these in a storage device such as a hard disk provided in the compute. The computer includes general-purpose PC, smartphone and tablet-type terminal device.

Furthermore, the program according to the example embodiment may be executed by a computer system constructed with a plurality of computers. In this case, for example, each computer may function as one of the learning model selection unit 11, the estimation unit 12, the presentation unit 13, and the learning model generation unit 14.

### Physical Configuration

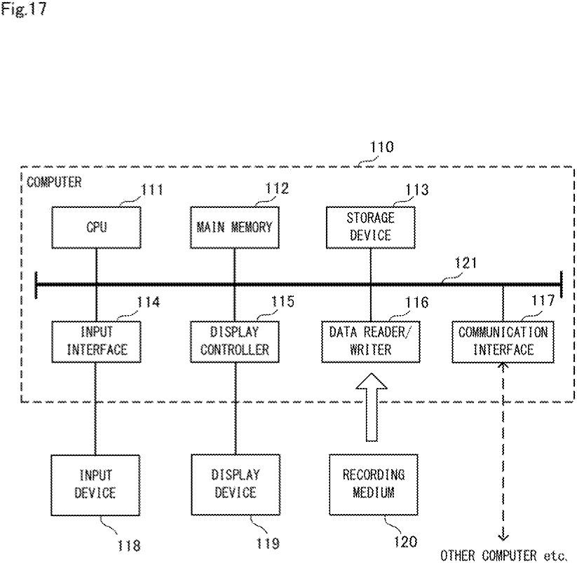

Using FIG. 17, the following describes a computer that realizes the diagnosis assistance apparatus by executing the program according to the example embodiment. FIG. 17 is a block diagram illustrating an example of a computer that realizes the diagnosis assistance apparatus according to the example embodiment.

As shown in FIG. 17, a computer 110 includes a CPU (Central Processing Unit) 111, a main memory 112, a storage device 113, an input interface 114, a display controller 115, a data reader/writer 116, and a communication interface 117. These components are connected in such a manner that they can perform data communication with one another via a bus 121.

The computer 110 may include a GPU (Graphics Processing Unit) or an FPGA (Field-Programmable Gate Array) in addition to the CPU 111, or in place of the CPU 111. In this case, the GPU or the FPGA can execute the programs according to the example embodiment.

The CPU 111 deploys the program (codes) according to the example embodiment, which is composed of a code group stored in the storage device 113 to the main memory 112, and carries out various types of calculation by executing the codes in a predetermined order. The main memory 112 is typically a volatile storage device, such as a DRAM (dynamic random-access memory).

Also, the program according to the example embodiment is provided in a state where it is stored in a computer-readable recording medium 120. Note that the program according to the present example embodiment may be distributed over the Internet connected via the communication interface 117.

Also, specific examples of the storage device 113 include a hard disk drive and a semiconductor storage device, such as a flash memory. The input interface 114 mediates data transmission between the CPU 111 and an input device 118, such as a keyboard and a mouse. The display controller 115 is connected to a display device 119, and controls display on the display device 119.

The data reader/writer 116 mediates data transmission between the CPU 111 and the recording medium 120, reads out the program from the recording medium 120, and writes the result of processing in the computer 110 to the recording medium 120. The communication interface 117 mediates data transmission between the CPU 111 and another computer.

Specific examples of the recording medium 120 include: a general-purpose semiconductor storage device, such as CF (CompactFlash®) and SD (Secure Digital); a magnetic recording medium, such as a flexible disk; and an optical recording medium, such as a CD-ROM (Compact Disk Read Only Memory).

Note that the diagnosis assistance apparatus 10 according to the can also be realized by using items of hardware that respectively correspond to the components, such as a circuit, rather than the computer in which the program is installed. Furthermore, a part of the diagnosis assistance apparatus 10 according to the example embodiment may be realized by the program, and the remaining part of the diagnosis assistance apparatus 10 may be realized by hardware.

A part or an entirety of the above-described example embodiment can be represented by (Supplementary Note 1) to (Supplementary Note 36) described below but is not limited to the description below.

### Supplementary Note 1

A diagnosis assistance apparatus, comprising:
a learning model selection unit that selects a first learning model in accordance with a patient to be diagnosed, the first learning model indicating a relationship between waveforms of an electrocardiogram and a disease;
an estimation unit that estimates, using the selected first learning model, a possibility of a disease of the patient to be diagnosed based on electrocardiogram data of the patient to be diagnosed; and
a presentation unit that presents a result of the estimation and an evidence for the result of the estimation.

### Supplementary Note 2

The diagnosis assistance apparatus according to Supplementary Note 1, wherein

using a second learning model indicating a correspondence relationship between information of patients and first learning models, the learning model selection unit selects one of the first learning models based on information of the patient to be diagnosed.

### Supplementary Note 3

The diagnosis assistance apparatus according to Supplementary Note 1 or 2, wherein

the estimation unit estimates the possibility of the disease of the patient to be diagnosed based on output results of the first learning model that respectively correspond to pieces of partial electrocardiogram data obtained by dividing the electrocardiogram data of the patient to be diagnosed at a predetermined time interval.

### Supplementary Note 4

The diagnosis assistance apparatus according to Supplementary Note 3, wherein

the estimation unit analyzes the possibility of the disease of the patient to be diagnosed based on results of analyzing the respective pieces of partial electrocardiogram data.

### Supplementary Note 5

The diagnosis assistance apparatus according to any one of Supplementary Notes 1 to 4, wherein

the result of the estimation includes the disease.

### Supplementary Note 6

The diagnosis assistance apparatus according to any one of Supplementary Notes 1 to 5, wherein

the evidence includes a ground based on which the disease has been specified.

### Supplementary Note 7

The diagnosis assistance apparatus according to Supplementary Note 6, wherein

the evidence is at least one of the electrocardiogram data and attributes of the patient to be diagnosed.

### Supplementary Note 8

The diagnosis assistance apparatus according to Supplementary Note 7, wherein
the learning model selection unit selects the first learning model based on the attributes of the patient to be diagnosed, and
the attributes are attributes corresponding to the selected first learning model.

### Supplementary Note 9

The diagnosis assistance apparatus according to any one of Supplementary Notes 1 to 8, wherein

the presentation unit presents the evidence, and presents the result of the estimation in accordance with a request for presenting the result of the estimation with respect to the presented evidence.

### Supplementary Note 10

The diagnosis assistance apparatus according to any one of Supplementary Notes 1 to 9, further comprising

a learning model generation unit that generates the first learning model through machine learning while using information of individuals, pieces of electrocardiogram data of the individuals, and labels indicating diseases corresponding to the pieces of electrocardiogram data as training data.

### Supplementary Note 11

The diagnosis assistance apparatus according to Supplementary Note 10, wherein

the learning model generation unit generates a second learning model through machine learning while using an output result from the first learning model corresponding to the pieces of electrocardiogram data of the individuals, the information of the individuals, and the labels indicating the diseases corresponding to the pieces of electrocardiogram data as training data.

### Supplementary Note 12

The diagnosis assistance apparatus according to Supplementary Note 11, wherein
the learning model generation unit
   specifies a first learning model corresponding to an individual using the second learning model based on information of the individual used as the training data, and
   updates the first learning model using electrocardiogram data and labels that have been selected as being correspondent to the specified first learning model among the pieces of electrocardiogram data of the individuals and the labels indicating the diseases corresponding to the pieces of electrocardiogram data used as the training data.

### Supplementary Note 13

A diagnosis assistance method, comprising:
a learning model selection step of selecting a first learning model in accordance with a patient to be diagnosed, the first learning model indicating a relationship between waveforms of an electrocardiogram and a disease;
an estimation step of estimating a possibility of a disease of the patient to be diagnosed based on electrocardiogram data of the patient to be diagnosed, using the selected first learning model; and
a presentation step of presenting a result of the estimation and an evidence for the result of the estimation.

### Supplementary Note 14

The diagnosis assistance method according to Supplementary Note 13, wherein

in the first learning model selection step, using a second learning model indicating a correspondence relationship between information of patients and first learning models, one of the first learning models is selected based on information of the patient to be diagnosed.

### Supplementary Note 15

The diagnosis assistance method according to Supplementary Note 13 or 14, wherein

in the estimating step, the possibility of the disease of the patient to be diagnosed is estimated based on output results of the first learning model that respectively correspond to pieces of partial electrocardiogram data obtained by dividing the electrocardiogram data of the patient to be diagnosed at a predetermined time interval.

### Supplementary Note 16

The diagnosis assistance method according to Supplementary Note 15, wherein

in the estimating step, the possibility of the disease of the patient to be diagnosed is analyzed based on results of analyzing the respective pieces of partial electrocardiogram data.

### Supplementary Note 17

The diagnosis assistance method according to any one of Supplementary Notes 13 to 16, wherein

the result of the estimation includes the disease.

### Supplementary Note 18

The diagnosis assistance method according to any one of Supplementary Notes 13 to 17, wherein

the evidence includes a ground based on which the disease has been specified.

### Supplementary Note 19

The diagnosis assistance method according to Supplementary Note 18, wherein

the evidence is at least one of the electrocardiogram data and attributes of the patient to be diagnosed.

### Supplementary Note 20

The diagnosis assistance method according to Supplementary Note 19, wherein
In the learning model selection step, the first learning model is selected based on the attributes of the patient to be diagnosed, and
the attributes are attributes corresponding to the selected first learning model.

### Supplementary Note 21

The diagnosis assistance method according to any one of Supplementary Notes 13 to 20, wherein

in the presenting step, the evidence is presented, and the result of the estimation is presented in accordance with a request for presenting the result of the estimation with respect to the presented evidence.

### Supplementary Note 22

The diagnosis assistance method according to any one of Supplementary Notes 13 to 21, further comprising

a learning model generation step of generating the first learning model through machine learning while using information of individuals, pieces of electrocardiogram data of the individuals, and labels indicating diseases corresponding to the pieces of electrocardiogram data as training data.

### Supplementary Note 23

The diagnosis assistance method according to Supplementary Note 22, wherein

in the generation of the learning model, a second learning model is generated through machine learning while using an output result from the first learning model corresponding to the pieces of electrocardiogram data of the individuals, the information of the individuals, and the labels indicating the diseases corresponding to the pieces of electrocardiogram data as training data.

### Supplementary Note 24

The diagnosis assistance method according to Supplementary Note 23, wherein
in the generation of the learning models,
   a first learning model corresponding to an individual is specified using the second learning model based on information of the individual used as the training data, and
   the first learning model is updated using electrocardiogram data and labels that have been selected as being correspondent to the specified first learning model among the pieces of electrocardiogram data of the individuals and the labels indicating the diseases corresponding to the pieces of electrocardiogram data used as the training data.

### Supplementary Note 25

A computer readable recording medium that includes a program recorded thereon, the program including instructions that cause a computer to carry out:
a learning model selection step of selecting a first learning model in accordance with a patient to be diagnosed, the first learning model indicating a relationship between waveforms of an electrocardiogram and a disease;
an estimation step of estimating a possibility of a disease of the patient to be diagnosed based on electrocardiogram data of the patient to be diagnosed, using the selected first learning model; and
a presentation step of presenting a result of the estimation and an evidence for the result of the estimation.

### Supplementary Note 26

The computer readable recording medium according to Supplementary Note 25, wherein

in the first learning model selection step, using a second learning model indicating a correspondence relationship between information of patients and first learning models, one of the first learning models is selected based on information of the patient to be diagnosed.

### Supplementary Note 27

The computer readable recording medium according to Supplementary Note 25 or 26, wherein

in the estimating step, the possibility of the disease of the patient to be diagnosed is estimated based on output results of the first learning model that respectively correspond to pieces of partial electrocardiogram data obtained by dividing the electrocardiogram data of the patient to be diagnosed at a predetermined time interval.

### Supplementary Note 28

The computer readable recording medium according to Supplementary Note 27, wherein

in the estimating step, the possibility of the disease of the patient to be diagnosed is analyzed based on results of analyzing the respective pieces of partial electrocardiogram data.

### Supplementary Note 29

The computer readable recording medium according to any one of Supplementary Notes 25 to 28, wherein

the result of the estimation includes the disease.

### Supplementary Note 30

The computer readable recording medium according to any one of Supplementary Notes 25 to 29, wherein

the evidence includes a ground based on which the disease has been specified.

### Supplementary Note 31

The computer readable recording medium according to Supplementary Note 30, wherein

the evidence is at least one of the electrocardiogram data and attributes of the patient to be diagnosed.

### Supplementary Note 32

The computer readable recording medium according to Supplementary Note 31, wherein
In the learning model selection step, the first learning model is selected based on the attributes of the patient to be diagnosed, and
the attributes are attributes corresponding to the selected first learning model.

### Supplementary Note 33

The computer readable recording medium according to any one of Supplementary Notes 25 to 32, wherein

in the presenting step, the evidence is presented, and the result of the estimation is presented in accordance with a request for presenting the result of the estimation with respect to the presented evidence.

### Supplementary Note 34

The computer readable recording medium according to any one of Supplementary Notes 25 to 33, wherein the program further including instructions that cause the computer to carry out:

a learning moder generation step of generating the first learning model through machine learning while using information of individuals, pieces of electrocardiogram data of the individuals, and labels indicating diseases corresponding to the pieces of electrocardiogram data as training data.

### Supplementary Note 35

The computer readable recording medium according to Supplementary Note 34, wherein

in the learning model generation step, a second learning model is generated through machine learning while using an output result from the first learning model corresponding to the pieces of electrocardiogram data of the individuals, the information of the individuals, and the labels indicating the diseases corresponding to the pieces of electrocardiogram data as training data.

### Supplementary Note 36

The computer readable recording medium according to Supplementary Note 35, wherein
in the generation of the learning models,
   a first learning model corresponding to an individual is specified using the second learning model based on information of the individual used as the training data, and
   the first learning model is updated using electrocardiogram data and labels that have been selected as being correspondent to the specified first learning model among the pieces of electrocardiogram data of the individuals and the labels indicating the diseases corresponding to the pieces of electrocardiogram data used as the training data.

Although the invention of the present application has been described above with reference to the example embodiment, the invention of the present application is not limited to the above-described example embodiment. Various changes that can be understood by a person skilled in the art within the scope of the invention of the present application can be made to the configuration and the details of the invention of the present application.

### INDUSTRIAL APPLICABILITY

As described above, according to the invention, it is possible to shorten a time period required for machine learning in machine learning of a parameter of a score function used in binary classification. The invention is useful in a variety of systems where binary classification is performed.

REFERENCE SIGNS LIST

| | |
|---|---|
| 10 | Diagnosis assistance apparatus |
| 11 | Learning model selection unit |
| 12 | Estimation unit |
| 13 | Presentation unit |
| 14 | Learning model generation unit |
| 15 | Storage unit |
| 16 | Selection model (Second learning model) |
| 17 | Disease estimation model (First learning model) |
| 20 | Display apparatus |
| 30 | Training data |
| 40 | Information (e.g., medical record data) of patient |
| 50 | Electrocardiogram data of the patient |
| 110 | Computer |
| 111 | CPU |
| 112 | Main memory |
| 113 | Storage device |
| 114 | Input interface |
| 115 | Display controller |
| 116 | Data reader/writer |
| 117 | Communication interface |
| 118 | Input device |
| 119 | Display device |
| 120 | Recording medium |
| 121 | Bus |

What is claimed is:

1. A diagnosis assistance apparatus, comprising:
at least one memory storing instructions; and
at least one processor configured to execute the instructions to:
selects a first learning model in accordance with a patient to be diagnosed, the first learning model indicating a relationship between waveforms of an electrocardiogram and a disease;
estimate, using the selected first learning model, a possibility of a disease of the patient to be diagnosed based on electrocardiogram data of the patient to be diagnosed; and
present a result of the estimation and an evidence for the result of the estimation.

2. The diagnosis assistance apparatus according to claim 1, wherein
further at least one processor configured to execute the instructions to:
using a second learning model indicating a correspondence relationship between information of patients and first learning models, select one of the first learning models based on information of the patient to be diagnosed.

3. The diagnosis assistance apparatus according to claim 1, wherein
further at least one processor configured to execute the
estimate the possibility of the disease of the patient to be diagnosed based on output results of the first learning model that respectively correspond to pieces of partial electrocardiogram data obtained by dividing the electrocardiogram data of the patient to be diagnosed at a predetermined time interval.

4. The diagnosis assistance apparatus according to claim 3, wherein
further at least one processor configured to execute the instructions to:
analyze the possibility of the disease of the patient to be diagnosed based on results of analyzing the respective pieces of partial electrocardiogram data.

5. The diagnosis assistance apparatus according to claim 1, wherein
the result of the estimation includes the disease.

6. The diagnosis assistance apparatus according toclaim 1, wherein
the evidence includes a ground based on which the disease has been specified.

7. The diagnosis assistance apparatus according to claim 6, wherein
the evidence is at least one of the electrocardiogram data and attributes of the patient to be diagnosed.

8. The diagnosis assistance apparatus according to claim 7, wherein
further at least one processor configured to execute the instructions to:
select the first learning model based on the attributes of the patient to be diagnosed, and
the attributes are attributes corresponding to the selected first learning model.

9. The diagnosis assistance apparatus according claim 1, wherein
further at least one processor configured to execute the instructions to:
present the evidence, and presents the result of the estimation in accordance with a request for presenting the result of the estimation with respect to the presented evidence.

10. The diagnosis assistance apparatus according to claim 1, further at least one processor configured to execute the instructions to:
generate the first learning model through machine learning while using information of individuals, pieces of electrocardiogram data of the individuals, and labels indicating diseases corresponding to the pieces of electrocardiogram data as training data.

11. The diagnosis assistance apparatus according to claim 10, wherein
further at least one processor configured to execute the instructions to:
generate a second learning model through machine learning while using an output result from the first learning model corresponding to the pieces of electrocardiogram data of the individuals, the information of the individuals, and the labels indicating the diseases corresponding to the pieces of electrocardiogram data as training data.

12. The diagnosis assistance apparatus according to claim 11, wherein
further at least one processor configured to execute the instructions to:
specify a first learning model corresponding to an individual using the second learning model based on information of the individual used as the training data, and
update the first learning model using electrocardiogram data and labels that have been selected as being correspondent to the specified first learning model among the pieces of electrocardiogram data of the individuals and the labels indicating the diseases corresponding to the pieces of electrocardiogram data used as the training data.

13. A diagnosis assistance method, comprising:
selecting a first learning model in accordance with a patient to be diagnosed, the first learning model indicating a relationship between waveforms of an electrocardiogram and a disease;
using the selected first learning model, estimating a possibility of a disease of the patient to be diagnosed based on electrocardiogram data of the patient to be diagnosed; and
presenting a result of the estimation and an evidence for the result of the estimation.

14-24. (canceled)

25. A non-transitory computer readable recording medium that includes a program recorded thereon, the program including instructions that cause a computer to carry out:
selecting a first learning model in accordance with a patient to be diagnosed, the first learning model indicating a relationship between waveforms of an electrocardiogram and a disease;
estimating a possibility of a disease of the patient to be diagnosed based on electrocardiogram data of the patient to be diagnosed, using the selected first learning model; and
presenting a result of the estimation and an evidence for the result of the estimation.
